(19) United States
(12) Patent Application Publication
(10) Pub. No.: US 2014/0257064 A1
(43) Pub. Date: Sep. 11, 2014

(54) SWEAT ANALYTE TESTING COMPONENTS AND METHODS

(71) Applicant: Birchwood Laboratories, Inc., Eden Prairie, MN (US)

(72) Inventors: Cydney A. Einck, Eden Prairie, MN (US); Nathan R. Erickson, Duluth, MN (US); Dennis Hepp, Coon Rapids, MN (US); Gary A. Messer, Coon Rapids, MN (US)

(73) Assignee: Birchwood Laboratories, Inc., Eden Prairie, MN (US)

(21) Appl. No.: 14/204,842

(22) Filed: Mar. 11, 2014

Related U.S. Application Data
(60) Provisional application No. 61/776,248, filed on Mar. 11, 2013.

(51) Int. Cl. A61B 5/00

(52) U.S. Cl. CPC A61B 5/4266
USPC 600/346

(57) ABSTRACT

Embodiments of the invention include adhesive electrode sets and related methods. In an embodiment, the invention includes an adhesive electrode set. The adhesive electrode set can include a first pad, a first electrode, a first electrical contact, a first conductive lead, a second pad, a second electrode, a second electrical contact, a second conductive lead, a flexible strip, and an adhesive material. The flexible strip can include a fold. The flexible strip can be configured to allow the distance between the first pad and the second pad to increase through flexion of the flexible strip. In an embodiment, the invention includes an analyte receiving test patch. The analyte receiving test patch can include a skin contact layer, a wick, an absorbent layer, a barrier film layer, and an adhesive frame. Other embodiments are also included herein.

This application claims the benefit of U.S. Provisional Application No. 61/776,248, filed Mar. 11, 2013, the contents of which are herein incorporated by reference.

### FIELD OF THE INVENTION

The present invention relates to sweat analyte testing components and related methods. More specifically, the present invention relates to sweat analyte testing components such as adhesive electrode sets, analyte receiving test patches, and related methods.

### BACKGROUND OF THE INVENTION

Certain types of diagnostic testing involve taking a sample of a tissue, a bodily fluid, or the like and then analyzing the sample for its properties such as the concentration of various components (naturally occurring or non-naturally occurring). By way of example testing for chloride concentration in the sweat of a newborn is a diagnostic technique for the early identification of cystic fibrosis. Testing for the presence of certain illicit compounds or metabolites in the sweat of a subject can be a diagnostic test for drug abuse.

Gathering sweat samples can pose various challenges. One challenge is how to stimulate the production of sweat. Another challenge is how to collect the sweat sample. Yet another challenge is how to process the sweat to determine the presence of and/or the amount of the analyte in the sweat sample.

### SUMMARY OF THE INVENTION

Embodiments of the invention include sweat analyte testing components and related methods. In an embodiment, the invention includes an adhesive electrode set. The adhesive electrode set can include a first pad, a first electrode, a first electrical contact, and a first conductive lead. The adhesive electrode set can also include a second pad, a second electrode, a second electrical contact, and a second conductive lead. The adhesive electrode set can also include a flexible strip, and an adhesive material. The first pad can include a first flexible substrate and a first aperture. The second pad can include a second flexible substrate and a second aperture. The flexible strip can include a fold. The first electrode can be aligned with the first aperture in the first pad. The first conductive lead can provide electrical communication between the first electrode and the first electrical contact. The second electrode can be aligned with the second aperture in the second pad. The second conductive lead can provide electrical communication between the second electrode and the second electrical contact. The flexible strip can connect the first pad and the second pad. The flexible strip can be attached to the first conductive lead and the second conductive lead. The flexible strip can be configured to allow the distance between the first pad and the second pad to increase through flexion of the flexible strip.

In an embodiment, the invention includes an analyte receiving test patch. The analyte receiving test patch can include a skin contact layer, a wick, an absorbent layer, a barrier film layer, and an adhesive frame. The skin contact layer can include an aperture. The barrier film layer can include a circular fill line. The wick can be configured to engage the aperture in the skin contact layer. The absorbent layer can be disposed over the skin contact layer. The barrier film layer can be disposed over the absorbent layer. The circular fill line can be imprinted thereon. The adhesive frame can be disposed over the barrier film layer.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE FIGURES

The invention may be more completely understood in connection with the following drawings, in which:

FIG. 1 is a schematic perspective view of an adhesive electrode set in accordance with various embodiments herein.

FIG. 2 is a schematic top view of an adhesive electrode set in an expanded configuration in accordance with various embodiments herein.

FIG. 3 is a schematic bottom view of an adhesive electrode set in an expanded configuration in accordance with various embodiments herein.

FIG. 4 is an exploded view of an adhesive electrode set in accordance with various embodiments herein.

FIG. 5 is a schematic top view of an adhesive electrode set in an expanded configuration in accordance with various embodiments herein.

FIG. 6 is a schematic perspective view of an analyte receiving test patch in accordance with various embodiments herein.

FIG. 7 is an exploded view of an analyte receiving test patch in accordance with various embodiments herein.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

### Adhesive Electrode Sets

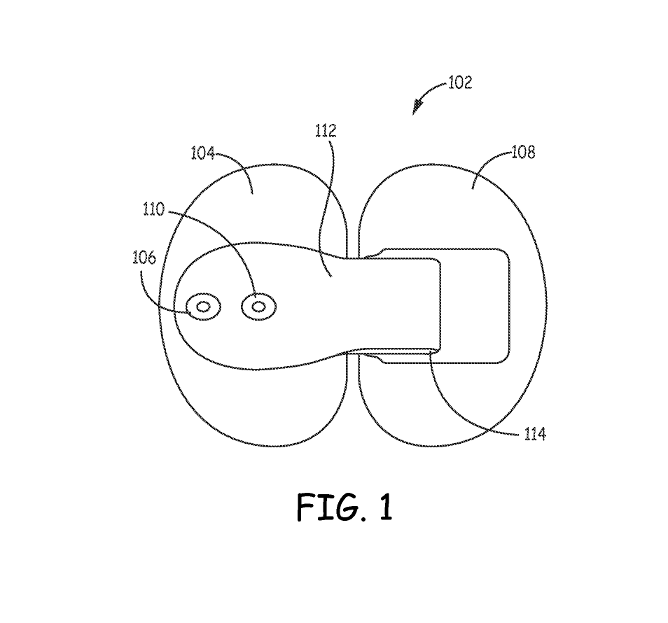

Embodiments herein can include sweat analyte testing components such as adhesive electrode sets, analyte receiving test patches, and related methods. Referring now to FIG. 1, an adhesive electrode set 102 includes a first pad 104 and a first electrical contact 106. The adhesive electrode set 102 also includes a second pad 108 and a second electrical contact 110. The adhesive electrode set 102 can include a flexible strip 112 interconnecting the first pad 104 and the second pad 108. The flexible strip 112 includes a fold 114. In some embodiments, the fold 114 can be a Z-fold.

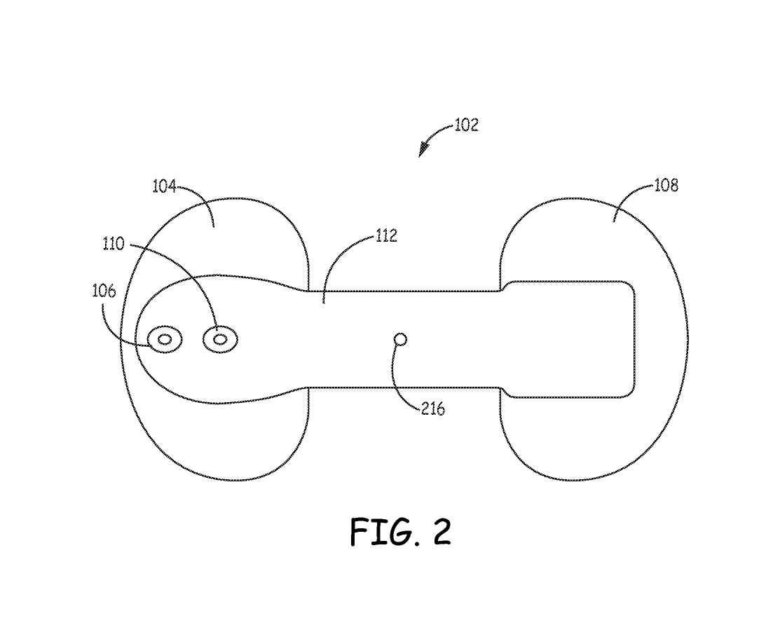

It will be appreciated that through unfolding of the fold 114, the distance between the first pad 104 and the second pad 108 can be changed. FIG. 1 shows the adhesive electrode set with the flexible strip folded so that the first pad 104 and the second pad 108 are immediately adjacent to one another. The adhesive electrode set can also assume an unfolded configuration where the first pad 104 and the second pad 108 are spaced apart from one another. Referring now to FIG. 2, the adhesive electrode set 102 includes a first pad 104, a first electrical contact 106, a second pad 108, a second electrical contact 110, and a flexible strip 112. The adhesive electrode set 102 can also include an adhesive material 216.

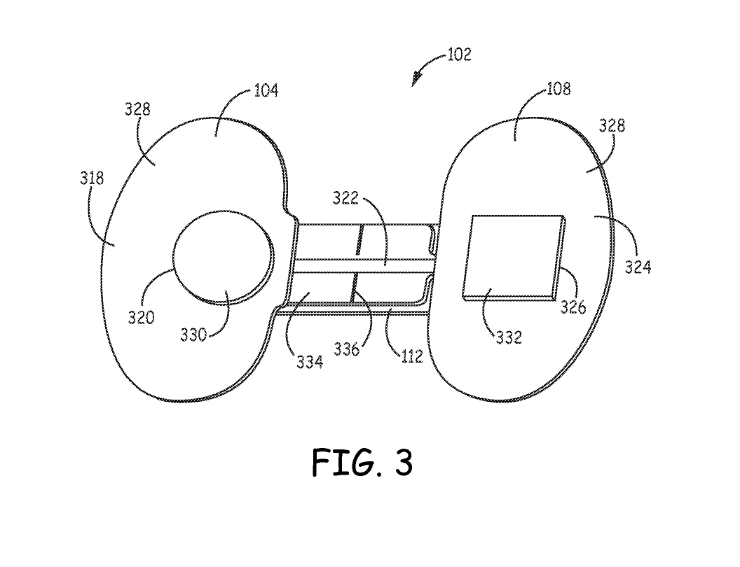

Referring now to FIG. 3, a schematic bottom view of an adhesive electrode set is shown in an expanded configuration in accordance with various embodiments herein. The adhesive electrode set 102 includes a first pad 104, a second conductive lead 322, a second pad 108, a flexible strip 112, and an adhesive material 328. The adhesive material 328 is disposed on the first pad 104 and second pad 108 and can serve to provide adhesive force between the pads and the skin of a test subject.

The first pad 104 includes a first flexible substrate 318 (such as a polymeric material) and a first aperture 320. The adhesive electrode set 102 can include a first hydrogel 330. The first hydrogel 330 can be aligned with the first aperture 320. The second pad 108 includes a second flexible substrate 324 and a second aperture 326. The adhesive electrode set 102 can include a second hydrogel 332. The second hydrogel 332 can be aligned with the second aperture 326. The flexible strip 112 can include a polyester backer 334. The polyester backer 334 can include a perforation line 336. The perforation line 336 can serve to provide a bending point on the polyester backer 334.

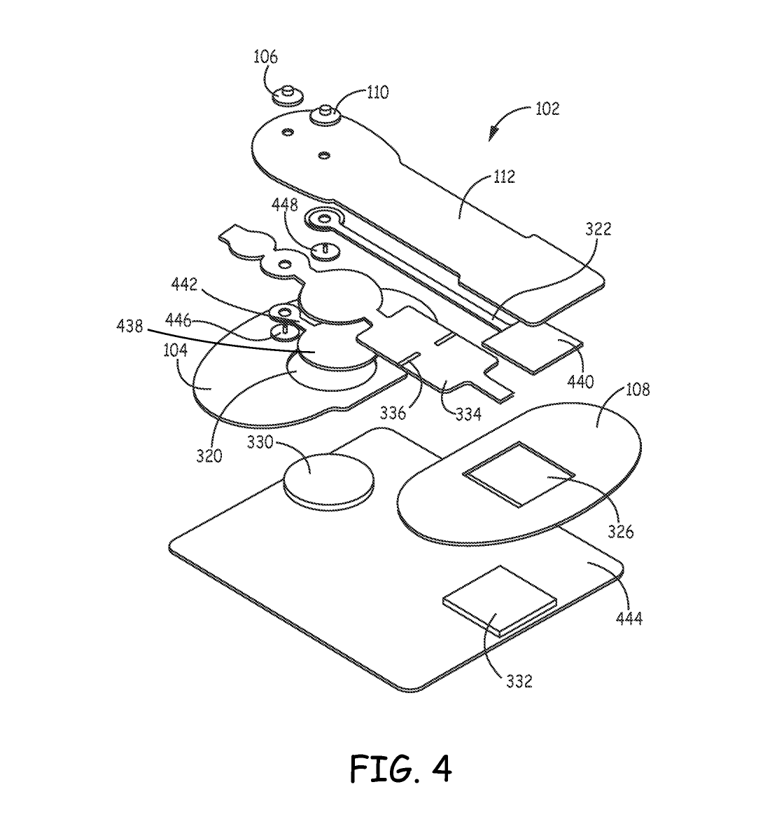

Referring now to FIG. 4, an exploded view of an adhesive electrode set is shown in accordance with various embodiments herein. The adhesive electrode set 102 includes a first pad 104, a first electrode 438, a first electrical contact 106, and a first conductive lead 442. The first conductive lead 442 and the first electrode 438 can be formed with a conductive ink. The adhesive electrode set 102 also includes a second pad 108, a second electrode 440, a second electrical contact 110, a second conductive lead 322, and a flexible strip 112. The second electrode 440 and the second conductive lead 322 can be formed with a conductive ink. The first pad 104 includes a first flexible substrate and a first aperture 320. The second pad 108 includes a second flexible substrate and a second aperture 326. The adhesive electrode set 102 can include a first hydrogel 330. The adhesive electrode set 102 can include a second hydrogel 332. The flexible strip 112 can include a polyester backer 334. The polyester backer 334 can include a perforation line 336. The adhesive electrode set can also include a first electrode post 446 and a second electrode post 448. The first electrode post 446 can contact the first conductive lead 442 and connect to the first electrical contact 106. The second electrode post 448 can contact the second conductive lead 322 and connect to the second electrical contact 110. The adhesive electrode set 102 can also include a liner 444. The liner 444 can include a material that releases easily from the adhesive under the first pad 104 and second pad 108. In this way, the liner 444 can protect the adhesive until the adhesive electrode set is ready for use and then the liner 444 can be easily removed to allow the adhesive electrode set to stick to the skin of a test subject.

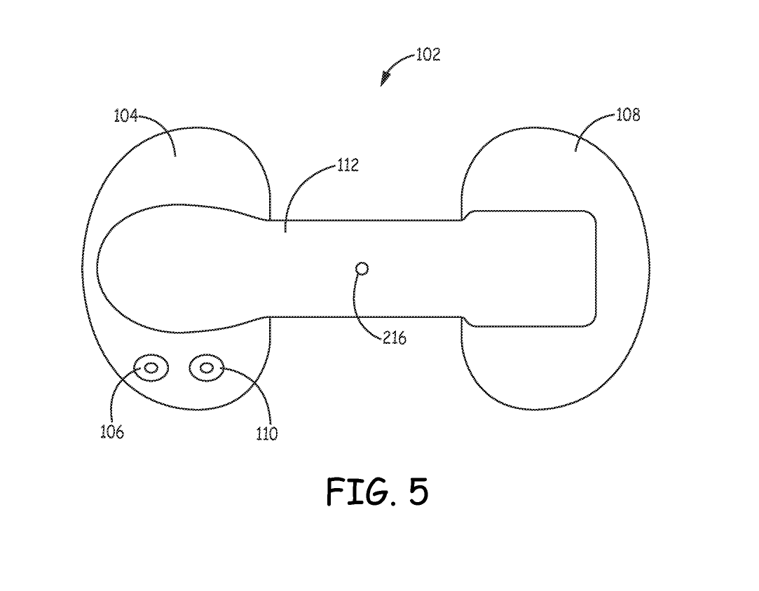

It will be appreciated that the electrical contacts can be located in various positions. Referring now to FIG. 5, a schematic top view of an adhesive electrode set is shown in an expanded configuration in accordance with various embodiments. In this view, the adhesive electrode set 102 includes a first pad 104, a first electrical contact 106, a second pad 108, a second electrical contact 110, and a flexible strip 112. The first electrical contact 106 and the second electrical contact 110 are disposed on the first pad 104. The adhesive electrode set 102 can include a second adhesive material 216.

In operation, a current/voltage source can be connected to the electrical contacts. The current can pass from first electrical contact through the first electrical lead and to the first electrode. From there, the current can pass into the first hydrogel and cause sweat inducing components therein (such as pilocarpine) to pass into the skin of the subject to be tested. The current moves through the tissue of the subject can back to the second hydrogel. From the second hydrogel the current passes into the second electrode, through the second electrical lead and back to the second electrical contact and the current/voltage source completing the circuit.

The first electrode can be aligned with the first aperture. In this way, the first electrode can be in the proper position to contact the first hydrogel. The first conductive lead can provide electrical communication between the first electrode and the first electrical contact. The second electrode can be aligned with the second aperture. In this way, the second electrode can be in the proper position to contact the second hydrogel. The second conductive lead can provide electrical communication between the second electrode and the second electrical contact.

The flexible strip can connect the first pad and the second pad. The flexible strip can be attached to the first conductive lead and the second conductive lead. The flexible strip can be configured to allow the distance between the first pad and the second pad to increase through flexion of the flexible strip and/or unfolding of the fold. The fold can be a Z-bend fold. In some embodiments, the distance between the first pad and the second pad can vary between 0 mm and 200 mm through unfolding of the fold. In some embodiments, the distance between the first pad and the second pad can be greater than about 10 mm. In some embodiments, the distance between the first pad and the second pad can be greater than about 23 mm. In some embodiments, the distance between the first pad and the second pad can be greater than about 36 mm. In some embodiments, the distance between the first pad and the second pad can be less than about 100 mm. In some embodiments, the distance between the first pad and the second pad can be less than about 83 mm. In some embodiments, the distance between the first pad and the second pad can be less than about 66 mm. In some embodiments, the distance between the first pad and the second pad can be between about 10 mm and about 100 mm. In some embodiments, the distance between the first pad and the second pad can be between about 23 mm and about 83 mm. In some embodiments, the distance between the first pad and the second pad can be between about 36 mm and about 66 mm.

The adhesive material disposed under the first pad and the second pad can be a biocompatible adhesive. In some embodiments, the adhesive material can be a biocompatible non-permanent adhesive. In some embodiments, the adhesive material can be an acrylic adhesive. In some embodiments, the adhesive under the pads and the adhesive on the flexible strip can be the same. In some embodiments, these adhesives are different.

In various embodiments the first conductive lead is disposed over the second conductive lead (such as shown in FIG. 5), but the first conductive lead is electrically isolated from the second conductive lead. This can be achieved in various ways. In some embodiments, a layer of a dielectric material is disposed between the first conductive lead and the second conductive lead. For example, a layer of a polymer (such as the polyester backer) can be used to electrically isolate the first conductive lead from the second conductive lead. In some embodiments, the first conductive lead and the second conductive lead can be formed from a conductive ink. For example, the conductive ink can be printed onto a polyester backer and then dried to form the conductive leads. Conductive inks are known in the art and can include various components. In some embodiments, the conductive ink can include silver chloride. Similarly, the first and second electrodes can be formed from a conductive ink by printing onto a substrate such as a polyester backer. However, it will be appreciated that in other embodiments conductive inks are not used and the conductive leads and/or electrodes are formed from electrically conductive materials such as metals or the like.

In some embodiments, the first electrical contact is not disposed directly over the first electrode. In some embodiments, the second electrical contact is not disposed over the second electrode. While not intending to be bound by theory, it has been discovered that placement of the electrical contact in a position that is not directly over the corresponding electrode results in a more desirable current distribution.

The pads can be formed of various materials. The first pad can be formed of a polymer, cellulosic material, fabric, woven, or nonwoven materials. In some embodiments, the first pad can be a polyurethane foam. The second pad can be formed of a polymer, cellulosic material, fabric, woven, or nonwoven materials. The second pad can be a polyurethane foam.

The adhesive electrode set can include a first hydrogel. The first hydrogel can include a sweat inducing agent. The sweat inducing agent can be pilocarpine nitrate. The adhesive electrode set can include a second hydrogel. The second hydrogel can be electrically conducive. In some embodiments, the second hydrogel can include a salt. The salt can include metal salts, base metal salts, sodium salts, or potassium salts. The salt can be potassium sulfate.

The first electrical contact and the second electrical contact can be any type of contact that is desired. In some embodiments, the first electrical contact and the second electrical contact can be a snap fitting. The first electrical contact and the second electrical contact can be formed of a metal or another suitable conductive material.

The flexible strip can include one or more polyester backers. In some embodiments, two polyester backers are included with each one serving as a substrate onto which an electrical lead is printed. The polyester backer(s) can include a perforation line. In some embodiments, the perforation line can be oriented perpendicular to the lengthwise axis of the polyester backer.

### Analyte Receiving Test Patches

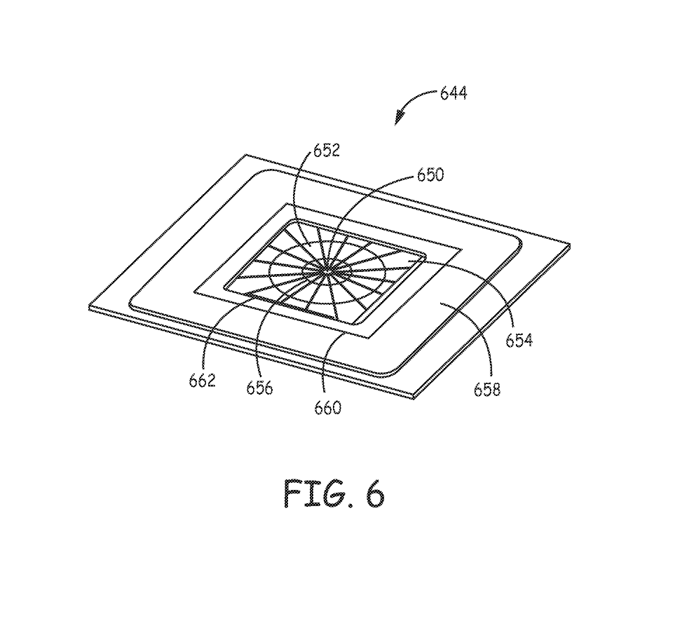

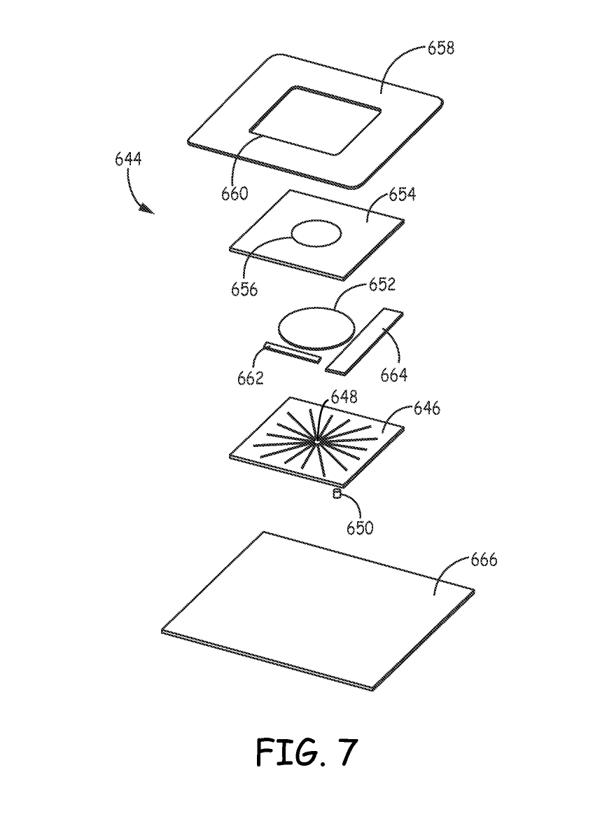

Analyte receiving test patches are also included in various embodiments herein. Referring now to FIG. 6, a schematic perspective view of an analyte receiving test patch is shown in accordance with various embodiments herein. The analyte receiving test patch 644 includes a skin contact layer 646 (shown in FIG. 7), a wick 650, an absorbent layer 652, a barrier film layer 654, and an adhesive frame 658. The barrier film layer 654 includes a circular fill line 656. The adhesive frame 658 can define an aperture 660. The analyte receiving test patch 644 can include a barcode panel 662.

Referring now to FIG. 7, an exploded view of an analyte receiving test patch is shown in accordance with various embodiments herein. The analyte receiving test patch 644 includes a skin contact layer 646, a wick 650, an absorbent layer 652, a barrier film layer 654, and an adhesive frame 658. The skin contact layer 646 includes an aperture 648. The barrier film layer 654 includes a circular fill line 656. The circular fill line 656 can be imprinted on the barrier film layer 654. The adhesive frame 658 can define an aperture 660. The adhesive frame 658 can overlap other components and allow for the test patch to be adhered to the skin of a test subject. The analyte receiving test patch 644 can also include barcode panel 662. The analyte receiving test patch can also include tab 664. The analyte receiving test patch 644 can also include a liner 666. The liner 666 can include a material that releases easily from the adhesive under the adhesive frame 658. In this way, the liner 666 can protect the adhesive until the patch is ready for use and then the liner 666 can be easily removed to allow the patch to stick to the skin of a test subject.

In operation, the analyte receiving test patch is applied to the skin of a subject to be tested after a sweat inducing agent has been delivered transdermally. The sweat is generated and is collected into optional channels formed into the bottom surface of the skin contact layer. The sweat passes to the wick and is transferred up through the aperture in the skin contact layer and passes into the absorbent layer. The sweat causes a reaction with components in the absorbent layer resulting in a visible change to allow a user to see how far sweat has penetrated through the absorbent layer. The user waits until the sweat reaches the fill line imprinted onto the barrier film layer. Then the adhesive frame and the barrier film layer are removed which allows the sweat to dry and prevents further migration of the sweat through the absorbent layer.

The wick can be configured to engage the aperture in the skin contact layer. The wick can have properties conducive to wicking of sweat. The wick can be porous material.

The skin contact layer can include polymer. The polymer can be fluid impermeable polymer. The polymer can be biocompatible polymer.

The absorbent layer can be disposed over the skin contact layer. The absorbent layer can be porous material. The absorbent layer can be webbed material. The absorbent layer can be cellulosic material. The absorbent layer can be impregnated with silver chromate. The absorbent layer can be impregnated with phenol red.

The barrier film layer can be disposed over the absorbent layer. The barrier film layer can include polymer. The polymer can be fluid impermeable polymer. The polymer can be biocompatible polymer. In some embodiments, the barrier film can be configured to separate from the absorbent layer after sweat absorption to stop liquid migration. The circular fill line can be imprinted onto the barrier layer. In some embodiments, all points of the fill line are substantially equidistant from the aperture in the skin contact layer.

The adhesive frame can be disposed over the barrier film layer. The adhesive frame can include substrate defining an aperture. The adhesive frame can include a layer of acrylic adhesive. The adhesive can allow the adhesive frame to seal the barrier layer to the other components and the whole analyte receiving test patch to the skin of a test subject.

In some embodiments, the analyte receiving test patch can include a barcode panel. The barcode panel can include various pieces of information. In some embodiments, the barcode panel can include lot specific information. Lot specific information can include information such as calibration information specific to the lot.

In various embodiments, kits are included herein. By way of example, kits can include an adhesive electrode set and an analyte receiving patch. The adhesive electrode set can include a first pad, a first electrode, a first electrical contact, and a first conductive lead. The adhesive electrode set can also include a second pad, a second electrode, a second electrical contact, and a second conductive lead. The adhesive electrode set can also include a flexible strip, and an adhesive material. The analyte receiving test patch can include a skin contact layer, a wick, an absorbent layer, a barrier film layer, and an adhesive frame. The skin contact layer can include an aperture. The barrier film layer can include a circular fill line. The wick can be configured to engage the aperture in the skin contact layer. The absorbent layer can be disposed over the skin contact layer. The barrier film layer can be disposed over the absorbent layer. The circular fill line can be imprinted thereon. The adhesive frame can be disposed over the barrier film layer. Other components can be included within the kit. By way of example, the kit can include the hydrogels in some embodiments. In some embodiments, the kit can also include alcohol wipes.

In some embodiments, methods of making components such as adhesive electrode sets and/or analyte receiving patches are included herein. In the context of an adhesive electrode set, the method can include forming an aperture in a first pad and forming a second aperture in a second pad. The method can also include printing a first electrical lead and first electrode on a first backer. The method can also include printing a second electrical lead and a second electrode on a second backer. The method can include forming perforations in the first and/or second backers. The method can include connecting the first and second pads with a flexible strip. The method can also include forming a fold in the flexible strip. It will be appreciated that in various embodiments less than all of these operations are performed. In some embodiments, additional operations can be performed in addition to those described herein.

It should be noted that, as used in this specification and the appended claims, the singular forms ‘a,’ ‘an,’ and ‘the’ include plural referents unless the content clearly dictates otherwise. Thus, for example, reference to a composition containing ‘a compound’ includes a mixture of two or more compounds. It should also be noted that the term ‘or’ is generally employed in its sense including ‘and/or’ unless the content clearly dictates otherwise.

It should also be noted that, as used in this specification and the appended claims, the phrase ‘configured’ describes a system, apparatus, or other structure that is constructed or configured to perform a particular task or adopt a particular configuration to. The phrase ‘configured’ can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

1. An adhesive electrode set comprising:
a first pad, the pad comprising a flexible substrate and defining a first aperture;
a first electrode aligned with the first aperture;
a first electrical contact;
a first conductive lead providing electrical communication between the first electrode and the first electrical contact;
a second pad, the pad comprising a flexible substrate and defining a second aperture;
a second electrode aligned with the second aperture;
a second electrical contact;
a second conductive lead providing electrical communication between the second electrode and the second electrical contact;
a flexible strip connecting the first pad and the second pad, the first and second conductive leads attached to the flexible strip, the flexible strip comprising a fold and configured to allow the distance between the first pad and the second pad increase through unfolding of the fold in the flexible strip; and
an adhesive material disposed under the first pad and the second pad.

2. The adhesive electrode set of claim 1, wherein the first electrical contact and the second electrical contact are disposed together on top of the flexible strip.

3. The adhesive electrode set of claim 1, wherein the first electrical contact and the second electrical contact are disposed together on top of one of the first pad or the second pad.

4. The adhesive electrode set of claim 1, wherein the first conductive lead is disposed over the second conductive lead, but the first conductive lead is electrically isolated from the second conductive lead.

5. The adhesive electrode set of claim 1, wherein the first electrical contact is not disposed over the first electrode.

6. The adhesive electrode set of claim 1, wherein the second electrical contact is not disposed over the second electrode.

7. The adhesive electrode set of claim 1, the first pad comprising a polyurethane foam.

8. The adhesive electrode set of claim 1, the second pad comprising a polyurethane foam.

9. The adhesive electrode set of claim 1, the adhesive electrode set comprising a first hydrogel.

10. The adhesive electrode set of claim 1, the sweat inducing agent comprising pilocarpine nitrate.

11. The adhesive electrode set of claim 1, the adhesive electrode set comprising a second hydrogel.

12. The adhesive electrode set of claim 11, the second hydrogel comprising a salt.

13. The adhesive electrode set of claim 12, the salt comprising potassium sulfate.

14. The adhesive electrode set of claim 1, the first conductive lead and the second conductive lead comprising a conductive ink.

15. The adhesive electrode set of claim 1, the fold comprising a Z-bend fold.

16. The adhesive electrode set of claim 1, wherein the distance between the first pad and the second pad can vary between 0 mm and 200 mm through unfolding of the fold.

17. The adhesive electrode set of claim 1, the flexible strip comprising a polyester backer.

18. The adhesive electrode set of claim 1, the adhesive electrode set comprising a second adhesive material.

19. A kit comprising:
an adhesive electrode set comprising
a first pad, the pad comprising a flexible substrate and defining a first aperture;
a first electrode aligned with the first aperture;
a first electrical contact;
a first conductive lead providing electrical communication between the first electrode and the first electrical contact;
a second pad, the pad comprising a flexible substrate and defining a second aperture;
a second electrode aligned with the second aperture;
a second electrical contact;
a second conductive lead providing electrical communication between the second electrode and the second electrical contact;
a flexible strip connecting the first pad and the second pad, the first and second conductive leads attached to the flexible strip, the flexible strip comprising a fold and configured to allow the distance between the first pad and the second pad increase through unfolding of the fold in the flexible strip; and
an adhesive material disposed under the first pad and the second pad; and
a chloride test patch comprising
a skin contact layer comprising an aperture;
a wick configured to engage the aperture in the skin contact layer;
an absorbent layer disposed over the skin contact layer;
a barrier film layer disposed over the absorbent layer, the barrier film comprising a fill line imprinted thereon; and
an adhesive frame disposed over the barrier film layer.

20. The kit of claim 19, the absorbent layer comprising silver chromate.

21. The kit of claim 19, the absorbent layer comprising phenol red.

22. The kit of claim 19, wherein the barrier film configured to separate from the skin contact laminate and components after sweat absorption to stop liquid migration.

23. The kit of claim 19, the analyte receiving test patch comprising barcode panel.
